(19) United States
(12) Patent Application Publication
(10) Pub. No.: US 2011/0295223 A1
(43) Pub. Date: Dec. 1, 2011

(54) REUSABLE DIAPER

(76) Inventor: MICHELLE McCULLOUGH, Papillion, NE (US)

(21) Appl. No.: 13/118,167

(22) Filed: May 27, 2011

Related U.S. Application Data
(60) Provisional application No. 61/348,912, filed on May 27, 2010.

(51) Int. Cl. A61F 13/505

(52) U.S. Cl. 604/385.14

(57) ABSTRACT

A reusable diaper comprising: a waterproof cover; a removable cloth lining, where the cloth lining removably attaches inside the waterproof cover; an elastic waist band; a set of elastic leg bands, where the leg bands encircle a set of leg openings; and a set of adjustment straps affixed to the waterproof cover wherein the adjustment straps secure the reusable diaper around a midsection of a wearer and the set of adjustment straps may be removed from the waterproof cover. Each adjustment strap may include a snap at a first end, where the snap enables the removal of each strap and a Velcro hook pad at a second end. The waterproof cover may include a Velcro loop strip attached to a front portion to accommodate the Velcro hook pad on each adjustment strap. The cloth lining may be made from either cotton, microfiber or a combination thereof.

### CROSS REFERENCE TO OTHER APPLICATIONS

This application claims priority to U.S. Provisional Application Serial No. 61/348,912 filed on May 27, 2010.

### BACKGROUND OF THE INVENTION

1. Field of the Invention

The present invention relates to a reusable diaper, which may have a waterproof cover to prevent leakage on surrounding surfaces.

2. Description of Related Art

Babies bring joy and laughter to those who surround them. They are a symbol of purity and new beginnings Along with the joy and excitement associated with a baby is the reality of their messes. Diapers, wipes, bibs and spit-up cloths are accustomed items to parents and caretakers alike. A baby may use 5-8 diapers a day leading to up to almost 3000 diapers per year. All of these diapers lead to large amounts of waste and trash which in turn get dumped into local landfills. Since disposable diapers are not biodegradable, trash just builds and builds resulting in more landfills for future generations.

Cloth diapers provide a more environmentally friendly alternative to disposable diapers, but there is a major problem associated with their use. They leak. As soon as a baby urinates in the cloth diaper, it must quickly be changed to prevent another mess in the surrounding surfaces. This problem makes transporting the baby difficult, because during travel a baby's waste may spill on car seats, strollers and carriers. The parent's attention must always be focused on the bowel movements of the child and thus the nearest location to change them.

It would be beneficial to have a reusable diaper which prevents leaks for a little more peace of mind of the child's parents. Another desirable aspect would be to have a reusable diaper wherein the sides may be adjusted to fit the midsection of the baby.

### SUMMARY OF THE INVENTION

The present invention relates to a reusable diaper comprising: a waterproof cover; a removable cloth lining, where the cloth lining removably attaches inside the waterproof cover; an elastic waist band; a set of elastic leg bands, where the leg bands encircle a set of leg openings; and a set of adjustment straps affixed to the waterproof cover wherein the adjustment straps secure the reusable diaper around a midsection of a wearer and the set of adjustment straps may be removed from the waterproof cover. Each adjustment strap may include a snap at a first end, where the snap enables the removal of each strap and a Velcro hook pad at a second end. The waterproof cover may include a Velcro loop strip attached to a front portion to accommodate the Velcro hook pad on each adjustment strap. The cloth lining may be made from either cotton, microfiber or a combination thereof.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 is a perspective view of a reusable diaper in accordance with an embodiment of the present invention.

FIG. 2 is a perspective view of an inner portion of a reusable diaper in accordance with an embodiment of the present invention.

Like reference numerals refer to like parts throughout the description of several views of the drawings.

### DETAILED DESCRIPTION

The present invention provides a reusable diaper incorporating a cloth diaper with a waterproof outer cover. The reusable diaper may be environmentally friendly by eliminating the need for disposable diapers, thus reducing the amount of trash and waste produced. The reusable diaper may comprise a waterproof cover, which may prevent leaks and spills onto a surrounding surface. The waterproof cover may attach to a cloth lining, which may be removed for washing. The reusable diaper may have adjustment straps to fit around the midsection of the wearer, and the adjustment straps may be removed to prevent warping during washing.

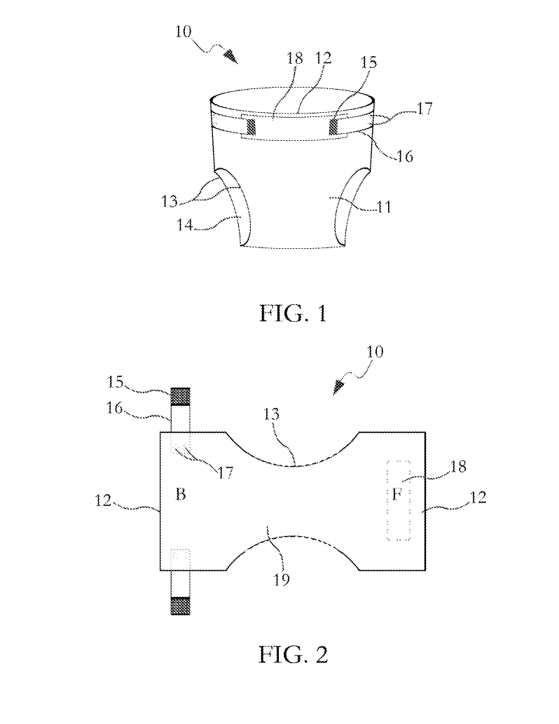

Turning descriptively to the drawings, referring to FIG. 1, a perspective view of a reusable diaper (10) in accordance with an exemplary embodiment of the present invention is shown. The reusable diaper (10) may comprise a waterproof outer cover (11), a waist band (12) and a set of leg bands (13) (hereinafter leg bands). The waterproof outer cover (11) may be made of plastic or another impermeable material to prevent moisture and liquid from leaking out of the reusable diaper (10). Graphics or pictures may be placed on the waterproof outer cover (11) to be aesthetically pleasing to either boys or girls. The waist band (12) may include an elastic strip to allow for a snug yet comfortable fit around a wearer's midsection. The leg bands (13) may encircle a set of leg openings (14) (hereinafter leg openings), and may fit around an upper portion of a wearer's leg. The leg bands (13) may also include an elastic strip to prevent liquid from leaking out of the leg openings (14).

The reusable diaper (10) may include a set of adjustment straps (16) (hereinafter adjustment straps) to secure the waist band (12) around the wearer's midsection. The adjustment straps (16) may be made of elastic or another type of stretchy material. At an end of the adjustment straps (16) there may be a Velcro hook pad (15). The reusable diaper (10) may have a Velcro loop strip (18) attached to a front portion (F). The Velcro hook pad (15) may adjustably attach the adjustment straps (16) to the Velcro loop strip (18). At an opposite end of the adjustment straps (16) may be a set of snaps (17) (hereinafter snaps) attached to a back portion of the reusable diaper (10). The snaps (17) may allow for the adjustment straps (16) to be removed during washing of the reusable diaper (10). By removing the adjustment straps (16), the Velcro hook pad (15) will not warp or be ruined during washing. The adjustment straps (16) combined with the Velcro hook pad (15) and the snaps (17) may permit the back portion of the reusable diaper to securely attach to the front portion of the reusable diaper along the Velcro loop strip (18) for a comfortable fit around the midsection of the wearer.

FIG. 2 illustrates a perspective view of an inner portion of the reusable diaper (10) in accordance with an embodiment of the present invention. FIG. 2 shows the reusable diaper (10) in an unfolded, flat position. The back portion (B) and the front portion (F) of the reusable diaper (10) are shown at opposite ends of the reusable diaper (10). The inner portion may have a cloth lining (19) made of cotton or a microfiber material. The cloth lining (19) may rest closest to the wearer's skin and may be soft and smooth for a comfortable feel. The cloth lining (19) may absorb any liquid or waste product produced by the wearer until the reusable diaper (10). Once soiled, the cloth lining (19) may be detached from the waterproof outer cover (11) to be washed and then replaced.

The reusable diaper may replace the need for disposable diapers, which create excess amounts of trash. Disposable diapers are wasteful because they are not biodegradable thus they build up in local landfills. Since the reusable diaper may be washed and reused they are environmentally friendly and are a “Green” alternative to disposable diapers. The waterproof cover allows the reusable diaper to keep any liquids in the diaper without creating a mess on any surrounding surfaces. The reusable diaper may be available in a variety of sizes. The sizes may range from those small enough to fit newborn babies to those large enough to fit an adult. Many adults require the need to use a diaper either because they may suffer from disabling medical condition or form incontinence. The reusable diaper (10) gives infants, adults and their caretakers an environmentally responsible alternative to disposable diapers, a comfortable fit by utilizing the adjustment straps and a cleaner improvement to readily available cloth diapers.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching.

What is claimed is:

1. A reusable diaper comprising:
a waterproof cover;
a removable cloth lining, where the cloth lining removably attaches inside the waterproof cover;
an elastic waist band;
a set of elastic leg bands, where the leg bands encircle a set of leg openings; and
a set of adjustment straps affixed to the waterproof cover wherein the adjustment straps secure the reusable diaper around a midsection of a wearer and the set of adjustment straps may be removed from the waterproof cover.

2. The reusable diaper of claim 1, wherein each adjustment strap includes a snap at a first end, where the snap enables the removal of each strap and a Velcro hook pad at a second end.

3. The reusable diaper of claim 2, wherein the waterproof cover has a Velcro loop strip attached to a front portion to accommodate the Velcro hook pad on each adjustment strap.

4. The reusable diaper of claim 1, wherein the cloth lining is made from either cotton, microfiber or a combination thereof.
